(19) United States
(12) Patent Application Publication
(10) Pub. No.: US 2004/0087852 A1
(43) Pub. Date: May 6, 2004

(54) Computer-assisted surgical positioning method and system

(76) Inventors: Edward Chen, Heidelberg (DE); Marwan Sati, Mississauga (CA); Haniel Croitoru, Toronto (CA); Peter Tate, Fergus (CA); Liqun Fu, Mississauga (CA)

Correspondence Address: PEARNE & GORDON LLP, 1801 EAST 9TH STREET, SUITE 1200, CLEVELAND, OH, 44114-3108, US

(21) Appl. No.: 10/467,445

(22) PCT Filed: Feb. 6, 2002
(86) PCT No.: PCT/CA02/00128

(30) Foreign Application Priority Data
Feb. 6, 2001 (CA) 2334495

(51) Int. Cl. A61B 5/05

(52) U.S. Cl. 600/407

(57) ABSTRACT

A method and apparatus are disclosed for defining a three-dimensional coordinate system in relation to an anatomical structure (64) for use with computer-assisted surgical systems. At least three landmarks (66, 68, 70) associated with the anatomical structure (64) are identified. Landmarks are identified using a digitization device (96) and their location refined using at least one single calibrated X-ray image. Using the landmarks (66, 68, 70), first and second planes are defined, the second plane being orthogonal to the first plane. These planes define a coordinate system, which can be used to ascertain the position trajectory of any object (80) with respect to the anatomical structure. Exposure to imaging radiation and additional surgical procedure is minimized.

### FIELD OF THE INVENTION

The present invention relates generally to computer-assisted surgical systems and, more particularly, to a method and system for defining a coordinate system in relation to an anatomical structure for use with computer-assisted surgical systems.

### BACKGROUND OF THE INVENTION

Severe damage to the hip joint caused by degeneration, trauma, disease or anatomical abnormalities make total hip replacements (THR) necessary.

A THR generally comprises four elements, which can be subdivided into two femoral components (femoral prosthesis shaft and head) and two acetabular components (acetabular ‘cup’ prosthesis and prosthesis inlay).

A successful THR procedure implies the selection of the right implant size through preoperative planning and correct intra-operative prostheses placement. Improper implant size and position can lead to hip joint dislocation, decreased range of motion and eventual loosening, failure of both the acetabular and femoral components, or the like. The objective for acetabular cup positioning methods is generally to achieve cup orientation angles of 15-20 degrees anteversion and 45 degrees inclination.

Correct conventional placement of the acetabular cup is surgically demanding due to the hemispherical acetabular shape and difficult anatomical landmark identification for alignment Limited surgical exposure of the patient and anatomical variations of the pelvis add to the complexity of the procedure.

A large number of non-computer-assisted instruments is known to facilitate the correct positioning of the acetabular cup by aligning posts—which are connected to the positioning rod holding the cup—with anatomical landmarks and external planes. Examples for this approach can be found in U.S. Pat. Nos. 4,305,394; 4,475,549; 4,994,064; 5,037,424; 5,061,270; 5,098,437; 5,116,339; 5,171,243; 5,250,051; 5,284,483; 5,320,625; 5,364,403; 5,527,317; 5,571,111; 5,584,837; 5,683,399; 5,755,794; 5,880,976; 5,954,727.

More recently, computer-assisted systems have been developed to facilitate the correct preoperative planning, cup positioning, femoral reaming, and the like. Most systems use tomographic patient imaging methods like CT and MRI to obtain anatomic patient data in digital form. Examples for CT based hip-joint planning and positioning systems are U.S. Pat. Nos.: 6,002,859 ; 5,995,738; 5,880,976, filed by DiGioia et al. In the above-mentioned systems, virtual patient models created using CT data are matched to the patient's anatomy using surface registration techniques in conjunction with an optical tracking system. In U.S. Pat. Nos. 5,251,127 and 5,305,203, issued to Raab, an electro-goniometer is used to digitize patient points in the CT data sets.

Other examples for CT based computer-assisted THR procedures are to be found in U.S. Pat. Nos. 5,086,401; 5,299,288 and 5,408,409, issued to Glassman et al. The above mentioned systems facilitate the robotic reaming of the femoral shaft. The patient-data-to-patient matching process is performed by artificial markers (fiducials) inserted into the patient's bones prior to the CT imaging and operation. Woolson (U.S. Pat. No. 5,007,936) uses three reference points on the acetabulum to be visually identified by the surgeon intra-operatively to match patient CT data.

The computer-assisted acetabular cup positioning devices described in the above references, have the following disadvantages:

1. Conventional cup positioning instruments, while being cost-effective, offer greater risk of inaccuracy due to the mere dependence on visual alignment by the surgeon.

2. Most computer-assisted-based procedures require additional pre-operative imaging necessary for trajectory-guidance purposes. Further, procedures requiring pre-operative placement of fiducials are an additional surgical operation. Such approaches result in increased costs, while additional operations and radiation also bear health risks for the patient.

Accordingly, there is a need for a method and system, which provides sufficient trajectory determination accuracy, without the need for additional pre-operative imaging and/or fiducial placement, or reliance upon visual identification. There is also interest to have a technology that obtains the trajectory information in a minimal invasive fashion to be used in minimal invasive surgical procedures.

### SUMMARY OF THE INVENTION

The present invention seeks to provide a method and system, which minimizes the above problems.

According to the invention, there is provided a method of defining a three-dimensional coordinate system using three landmarks associated with an anatomical structure that are digitized using a digitization device, and imaging to refine a location for these landmarks.

In an aspect of the invention, the landmarks are used to define a first vector and a second vector, which in turn are used to define the first plane and the second plane. The planes are used in defining the coordinate system. Geometrically, a “plane” is represented by a point in the plane and an orthogonal “normal vector” that points away from the plane forming a 90-degree angle between the plane and vector. Thus, either planes or vectors can be used to represent the coordinate system.

In another aspect of the invention, the landmarks are identified and location refined, using a digitization device and at least one calibrated X-ray image.

In another aspect of the invention, the coordinate system so defined is used to ascertain the position and alignment of an object with respect to the anatomical structure.

The invention defined above extends to all imaging modalities in computer-assisted, image-guided surgical navigation systems.

Also, according to the invention, there is provided a system for defining a coordinate system for computer-assisted surgical systems, means for using imaging to refine a location for the landmarks, including means for identifying at least three landmarks associated with the anatomical structure and means for defining the coordinate system using the landmarks.

Also, according to the invention, there is provided a computer-readable medium having computer-executable software code stored thereon, the code for defining a coordinate system for computer-assisted surgical systems, comprising a code for using imaging to refine a location for the landmarks for identifying three landmarks associated with the anatomical structure, and a code for defining a coordinate system using the landmarks.

A further aspect of the invention includes the identification of three pelvic landmarks used to define the frontal and sagittal plane of a pelvis. These planes are used to define a three-dimensional coordinate system for use in a computer-assisted surgical system. The pelvic locations of landmarks are identified using a digitization device and refined using at least one calibrated X-ray image. The coordinate system and the landmark locations therein are then available for use in the computer-assisted surgical system, including pre-operatively, for example, in diagnosis, surgical planning and design, three-dimensional modelling, virtual visualization and localization, surgical simulations, medical education, and the like, and intra-operatively, including in surgical navigation, instrument localization, positioning and tracking, visualization, and the like.

As a further aspect, the invention includes a method of ascertaining the trajectory of an object in relation to the defined coordinate system. Following definition of the coordinate system for a pelvis using the landmarks, a tracked object such as a tracked acetabular cup can be visualized, positioned, localized, and the like, in the computer-assisted surgical system, including in pre-operative procedures and intra-operative procedures such as acetabular cup positioning.

Advantageously, by defining the coordinate system in this fashion, exposure of patients to pre-operative imaging radiation is reduced and the need for additional surgical procedures such as fiducial placement is avoided, while providing sufficient imaging accuracy for computer-assisted surgical systems, including for acetabular cup positioning, in total hip replacement surgical procedures.

### BRIEF DESCRIPTION OF DRAWINGS

The present invention, by way of example only, will be further understood from the following description with references to the drawings in which:

FIG. 1 is a schematic layout of the system in accordance with an embodiment of the invention.

FIG. 2 is a representation of a frontal view of a pelvis.

FIG. 3 is a diagram of the coordinate system in accordance with an embodiment of the invention.

FIG. 4 is a representation of a tracked probe in accordance with an embodiment of the invention.

FIG. 5 is a diagram of an acetabular cup positioner in accordance with an embodiment of the invention.

FIG. 6 is a diagram of three orthogonal planes in accordance with an embodiment of the invention.

FIG. 7 is a diagram of a plane normal coordinate system in accordance with an embodiment of the invention.

### DETAILED DESCRIPTION OF THE PREFERRED EMBODIMENT

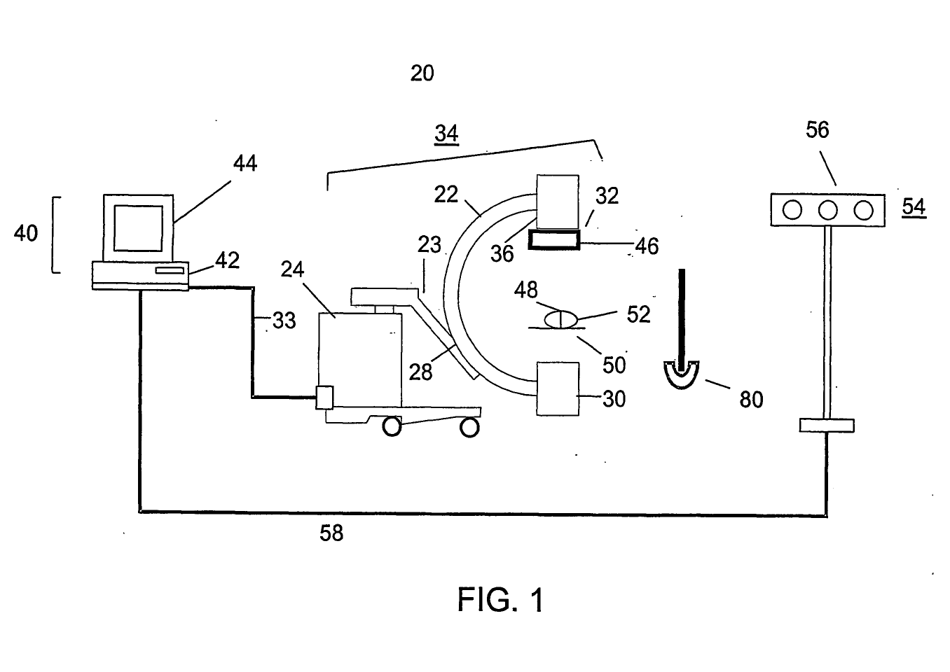

Referring to FIG. 1, a computer-assisted acetabular cup positioning apparatus includes a mobile fluoroscopic C-arm X-ray imaging device 20. Mobile X-ray devices used in the operating room are generally known as C-arms due to their shape. The imaging method is referred to as ‘fluoroscopy’ since no X-ray film is being used. Fluoroscopy-based navigation systems are commercially available and very common in operating rooms.

While the embodiment of the invention described is in reference to fluoroscopic-based navigation, it can be appreciated that other imaging modalities may be used, for example, computerized tomography (CT), magnetic resonance imaging (MRI, ultrasound, and bi-planar X-ray. However, for certain of these modalities, there is an additional need for pre-operative tomographic imaging, fiducial placement or intra-operative matching of tomographic datasets.

Imaging device 20 includes a C-arm 22 slidably and pivotally attached to a downwardly-extending L-arm 23 at an attachment point 28. The L-arm 23 is held in suspension by a mobile support base 24. The C-arm 22 is orbitable about an axis of orbital rotation, while the L-arm 23 is rotatable about an axis of lateral rotation to thereby rotate the C-arm 22 laterally.

X-ray source 30 is located at one end of C-arm 22 and X-ray image receptor assembly 32 is located at the other end of C-arm 22. The X-ray source 30 is capable of generating a continuous or pulsed stream of X-ray photons.

The C-arm 22, X-ray source 30 and image receptor assembly 32 are rotatable about and define a free space 34. Within the free space 34, an operating table 50 and patient 52 may be positioned. X-rays emitted from the X-ray source 30 passes through the free space 34 to the image receptor assembly 32, intersecting the patient 52, and generating a planar two-dimensional image of the patient. By orbitally and laterally rotating the C-arm 22 about the free space 34, X-rays may be directed to pass through the patient 52 along multiple planes to generate two-dimensional images from different perspectives.

The image receptor assembly 32 generates an image representing the intensities of received X-rays. In the preferred embodiment, the image receptor assembly 32 comprises an image intensifier 36 that converts the received X-ray photons to visible light. The image intensifier 36 is electronically coupled to a digital charge coupled device (CCD) camera (not shown) that converts the visible light to an analog video signal.

The image receptor assembly 32 may be additionally provided with an X-ray off detector (not shown) to detect when a new image has been inquired. For example, the X-ray detector may be in the form of a detector diode that directly absorbs received X-ray radiation or be a photodiode with a scintillator. The X-ray off detector may be used to synchronize the fluoroscopic image with the optical position tracking data as detailed below.

The image receptor assembly 32 is interfaced via electronic cables 33 to a computer system 40 to which imaging data is communicated.

The computer system 40 includes a computer 42 with a graphics processor. Preferably, the graphics processor is a video capture circuit board such as Matrox Meteor-II™ that is capable of capturing and digitizing an analog video signal. The computer 42 is electronically interfaced with at least one video display monitor 44 or other display via a video display card, for use in interactive viewing and display of images.

The computer system 40 is provided with a plurality of data input interfaces for the receipt, storage and processing of data received from external sources, as more particularly described below. Without limitation, input interfaces include electronic interfaces (for example, port connections to external source devices, modems, keyboard, mouse, etc.), optical interfaces, or radio frequency interfaces.

The computer system 40 is selected to be suitable for computer-assisted, image-guided surgery, including surgical navigation, diagnosis, surgical planning and design, three-dimensional modelling, virtual visualization and localization, surgical simulations, medical education, instrument localization and tracking, instrument positioning, and the like. For example, the computer system 40 is provided with sufficient memory, data storage, resolution, and processing speeds sufficient to calculate, process, store and display high quality, high volume, real-time images. The computer 42 may also be provided with a network card to interface with a network. Examples of computer systems are Dell™ Precision™ Workstations 330, 420 or 620.

The computer system 40 is further provided with software such as SNN Fluoro™ software, and the like, that allows for the acquisition and registration of fluoroscopic images and superimposition of optically-tracked instruments,.

The image receptor assembly 32 is further fitted with two calibration plates 46, which are clamped onto the image intensifier 36. The calibration plates 46 contains radio-opaque beads spaced in a well-defined geometry and are positioned adjacent to the image intensifier 36 in the path of incoming X-ray photons emitted from the X-ray source 30. The raw, unprocessed images as captured by the image intensifier 36 are overlaid with the images of the radio-opaque beads. The images of the beads will appear distorted from their true geometry following X-ray transmission through the calibration plate 46. Information regarding the actual positioning of the radio-opaque beads previously stored in the computer 42 is used in a mathematical model to compute image distortion.

The mathematical model is derived using conventional means and may be applied to process captured raw image for display in substantially distortion-free form. Distortion computation is described, for example, by Thomas S. Y. Tang, “Calibration and Point-Based Registration of Fluoroscopic Images”, Queen's University, Kingston, Ontario, Canada, January 1999 and Champleboux G, Lavallee S, Cinquin P “Accurate Calibration of Cameras and Range Imaging Sensors: The NPBS Method”, Proc. IEEE of Int. Conf. on Robotics and Automation, Nice France, 1992. The mathematical model may be embodied in software such as navigation or imaging software applications, and the like.

Alternatively, distortion may be corrected using alternate methods including methods dispensing with the need for one or both calibration plates.

In preoperative or non-operative procedures, such as surgical planning, diagnostics, simulation, or the like, the patient 52 is positioned in the free space 34, with the anatomical area of interest exposed. The patient is provided with a patient tracker 48.

Alternatively, a patient 52 is prepared for surgery within the free space 34, with the area of surgical interest exposed. When surgery commences, the patient 52 is provided with a patient tracker 48.

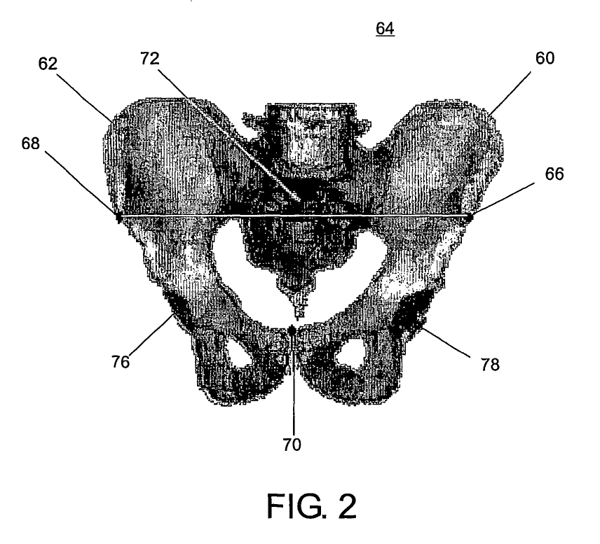

The patient tracker 48 is an active or passive optically-tracked instrument The patient tracker 48 is rigidly attached to the patient in close proximity to the interested area. In THR, the patient tracker 48 is attached to the frontal iliac crests of the pelvis 60 and 62, as indicated in FIG. 2, for example, using Kirschner wires which are drilled by the surgeon into the iliac crests 60 and 62. For accurate image guidance, the patient tracker 48 cannot be significantly moveable relative to the patient 52 and is preferably substantially immoveable; although, a less rigidly secured tracker may be acceptable in certain applications, for example, visualization and simulation. It will be appreciated that any movement of the patient tracker relative to the patient will affect the accuracy of any subsequent computation based on the location of the patient tracker 48.

The patient tracker 48 is used in conjunction with a position sensing system, as further described below. In the embodiment of FIG. 1, the patient tracker 48 is provided with a plurality of passive reflective disks or visible Light Emitting Diodes (LEDs) in a known geometry to yield orientation as well as positional information.

Alternatively, active optical trackers using infrared light emitting diodes (IREDs) may be attached onto the patient tracker 48. The trackers may be electronically connected onto a control unit of the position sensor system 54, which can control the emissions of the IREDs.

The position sensor 56 in the embodiment of FIG. 1 is an optical camera set a distance away from the imaging device, in unobstructed view of all trackers for which positional and orientational information is desired. The position sensor 56 uses triangulation and real time tracking algorithms to reconstructed three-dimensional coordinates of a tracker and is interfaced with the computer system 40 to communicate racking data. An example of a position sensor system 54 is the POLARIS™ system by Northern Digital Inc.

Alternate position sensor systems may be used. Active optical sensor systems may use IREDs as trackers. Hybrid position sensors trackers position and orientation of both active and passive trackers.

The position sensor 56 of the embodiment is interfaced via electronic cables 58 with the computer 42 for data communication.

The calibration plates 46 on the C-arm 22 are also provided with active or passive trackers, the position of which are tracked by the position sensor system 54, such that C-arm 22 positional information is communicated to the computer 42. Alternatively, active or passive trackers may be attached to predetermined positions on the image intensifier 36, elsewhere on the image receptor assembly 32, or other mobile portion of the C-arm 22.

The patient tracker 48 operates as a reference base attached to the patient 52 while at the same time the C-arm 22 position and orientation in space is also tracked by the position tracking system 54. The patient tracker 48 and the C-arm 22 trackers provide positional reference data for use with surgical navigation software, and the like.

The function of the patient tracker 48 is to determine the transformation between image coordinate and world coordinate systems (ie. the actual coordinates of objects in the operating room). These transformations are necessary to render optically-tracked instruments (such as drill guides, probes, awls, etc.) on the fluoroscopic image in the correct anatomical position. The process of image registration is known and, for example, is described in U.S. Pat. No. 5,772,594 titled “Fluoroscopic image guided orthopaedic surgery system with intra operative registration” issued to Barrack, E. F. on Jun. 30, 1998, and in Thomas S. Y. Tang, “Calibration and Point-Based Registration of Fluoroscopic Images”, Queen's University, Kingston, Ontario, Canada, January 1999.

While optical sensors are preferred as position sensors, other position sensors may be used, including mechanical sensors comprising articulated arms with potentiometers at each joint, sonic sensors comprising the detection of the speed and direction of soundwaves from positioned acoustic emitters, or magnetic sensors, which detect phase and intensity of magnetic fields.

Preferably, the position sensor system 54 is also capable of localizing in space (tracking) the position of surgical instrumentation and tools during intra-operative surgical procedure.

A tracker is a device that tracks the position and orientation of a rigid body. A tracker can include several components, for example, optical or acoustical markers that are used to determine a rigid object's position and orientation in space.

Tracked surgical instrumentation and tools, which include probes, pointers, wands, drill guides, awls, suction units with inserts, reference clamps and pins, may be provided with integrated tracking technology embedded in the tool, or permanently or temporarily mounted with one or more tracker components. Preferably, at least two tracker components are provided on tracked surgical instruments so as to permit tool orientation, as well as position, to be determined. Additional position trackers, active or passive, maybe temporarily attached to various objects in the operating room for positioning and reference purposes, for example, on the patient table.

Alternatively, additional position sensor systems (active optical sensors, sonic, mechanical, magnetic, radio frequency, etc.) may be used to separately track various tools or reference objects in the operating room. Such position sensors would also be interfaced with a computer system 40 provided with surgical navigation software.

The positioning of the tracked tools and positional trackers are pre-registered into the surgical navigation system prior to intra-operative use by conventional means.

Once the patient 52 within the free space 34 is fitted with the patient tracker 48, reference points, or landmarks, are identified to define the frontal plane of the patient. The landmarks may be anatomically significant structures, points, virtual points, prominences, and the like, suitable for plane definition and fast identification by the user. With reference to the human pelvis 64, the frontal plane of a person standing in upright position is defined by three landmarks on the pelvis (FIG. 2): left anterior superior iliac spine 66, right anterior superior iliac spine 68 and the centre of the pubis symphysis 70. As will be appreciated by persons skilled in the art, other landmarks may be ascertained and used to define the frontal or other planes for other anatomical structures, including other ball and socket joints on human, mammalian or other vertebrates.

For an accurate definition of the frontal plane of the human pelvis, it is preferred that both anterior superior iliac spine landmarks 66 and 68 are on the same height level in the anterior posterior and in the sagittal planes, and that the landmark on the pubic symphysis 66 is centred in the anterior posterior plane, as depicted in FIG. 2.

The three landmarks can be substantially identified by palpation by the surgeon, or other practitioner familiar with anatomy, using known techniques. The user then uses digitization devices such as a tracked probe 96 to determine the three-dimensional coordinate position of the three landmarks 66, 68 and 70. Preferably, the tracked probe 96 is a needle pointer capable of piercing the skin to contact the underlying bone. The needle pointer 96 has a tracking component 98 attached to the handle 100 of the probe. The location and orientation of the trajectory of the tip 102 of the needle relative to the tracking element 98 is known and communicated to the navigation software.

Using imaging techniques, the digitized positions of landmarks are refined in order to compensate for any inaccuracies in the user's location of the landmarks. For example, a single anterior-posterior X-ray view of the interested anatomical area is taken and the digitized position of a landmark within the plane normal to the X-ray beam direction can be adjusted in imaging software, or the like, accordingly. More particularly, the digitized position of the landmarks as displayed on the computer display 44 may be adjusted (ie. left, right, up or down) relative to the planar X-ray image, without modifying the depth of the point so as to coincide therewith. For example, the rough positions of the left anterior superior iliac spine 66, right anterior superior iliac spine 68, and the centre of pubis symphysis 70, obtained with the digitizing instrument, are adjustable to overlay the X-ray image of the landmarks. Alternatively, lateral or other X-ray views may also be used for fine adjustment depending on the procedure to the patient. In the further alternative, ultrasound imaging may be used for refining landmark coordinates.

Additional fluoroscopy images of the landmarks may be taken with the C-arm 22 rotated such that images are obtained of the interested area on two image planes for bi-planar X-ray reconstruction. This bi-planar method may be necessary if the surgical draping or large amount of soft tissue does not allow direct palpation of the landmark. Preferably, only two images are taken. However, depending on the size of the interested area, the size of the patient, and the diameter of the C-arm 22 imaging field, additional images may be required in order to capture all landmarks in fluoroscopic images. For example, where the field of view of the fluoroscopic image is identical to that of a plain radiograph, and the size of the pelvis is greater than can be captured in one image, each landmark can be selected with one image in the anterior posterior orientation and by one taken laterally, for a total of six planar fluoroscopic images.

The fluoroscopic images captured by the image intensifier 36 are communicated to the computer system 40 where they are corrected for distortion and stored for later use, for example, in surgical navigation to verify the location of the identified landmarks.

Intra-operatively, a procedure utilizing a single anterior-posterior X-ray image taken from the appropriate direction for refining landmark coordinates is preferred, as good quality lateral images are difficult and cumbersome to obtain, and reduced exposure of the patient to X-ray radiation is physiologically preferred.

Once the frontal plane is determined, the sagittal plane and the axial plane can be determined. When the coordinate location of landmarks 66, 68 and 70 are digitized and inputted into the imaging system, refined using the X-ray or fluoroscopic or other images, if taken, the cross product of the vector from landmarks 66 and 70 and the vector from landmarks 68 and 70 are used to define the frontal plane with the normal (frontal normal) pointing ahead of the patient 52. The midpoint 72 between 66 and 68 is then computed.

The sagittal plane is defined as the cross product of the vector from landmarks 70 and midpoint 72 and the normal vector of the frontal plane (frontal normal). The normal of the sagittal plane (sagittal normal) points to the left side of the patient.

The axial plane is defined as by the cross product of the sagittal normal and the frontal normal and its normal points towards the head of the patient.

The computations to determine the frontal plane, the sagittal planes and the axial planes are made, stored and applied in navigation software or the like. Such computations may be made prior to surgery as well as during the operative procedure.

For the human pelvis in THR surgery, the acetabular cup prosthetic is preferably orientated with angles of 15-20 degrees anteversion and 45 degrees inclination for human patients. A number of considerations is involved in the selection of the targeted angles of approach including: a desire to obtain a maximum range of motion, to achieve a minimum residual pain, to avoid impingement between the femur and other osseous structures in the pelvis, to avoid subsequent dislocation of the joint, and to otherwise avoid the need for a subsequent hip replacement due to improper placement of the prosthesis.

The anteversion and inclination angles are measured relative to the frontal, sagittal and axial planes. These planes are measured for the whole pelvis and the acetabular cup is placed relative to these planes. With reference to the left or right acetabulum 76 or 78 on the human pelvis, the targeted acetabular cup inclination angle of 45 degrees is projected on the frontal plane. An acetabular cup anteversion angle of 15 degrees is projected on the sagittal plane, which lies perpendicular to the frontal plane.

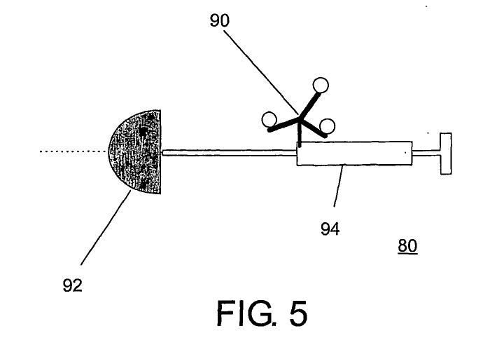

Referring to FIG. 5, a conventional acetabular cup positioner 80, for example, Zimmer™ Trilogy™ Acetabular Cup, equipped with a tracking element 90 on the reamer 94, is used to compute the acetabular cup position in relation to the patient's pelvic girdle. Preferably, active or passive optical tracking elements are attached to the reamer 94 of the cup positioner 80. The location and orientation of the cup trajectory relative to the tracking element 90 is known, and registered.

Using the optical position sensor system 54, or an alternative position sensing system, the position of the tracked acetabular cup positioner 80 relative to the patient tracker 48, and other tracked objects in the operating room, is communicated to the computer system 40 and displayed to the surgeon through the computer display 44, or other means, via navigation software, preferably in real-time.

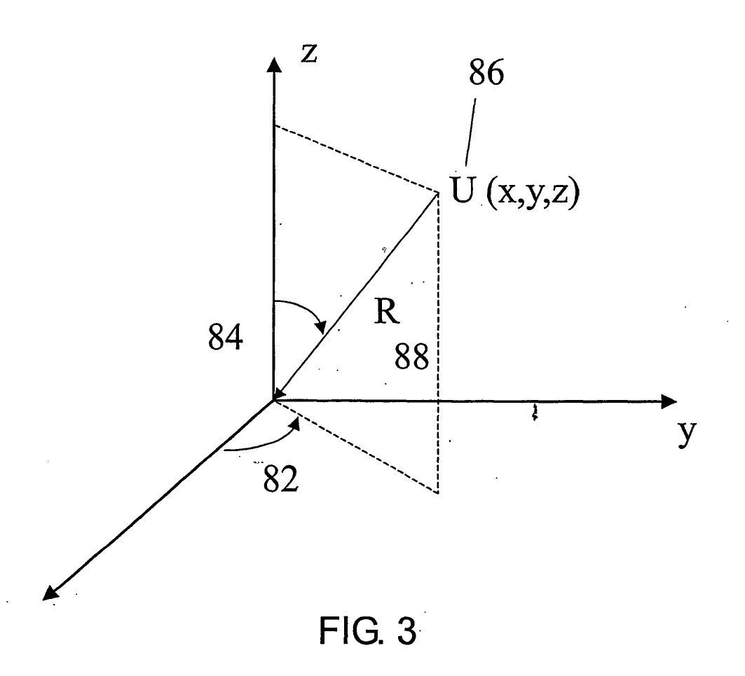

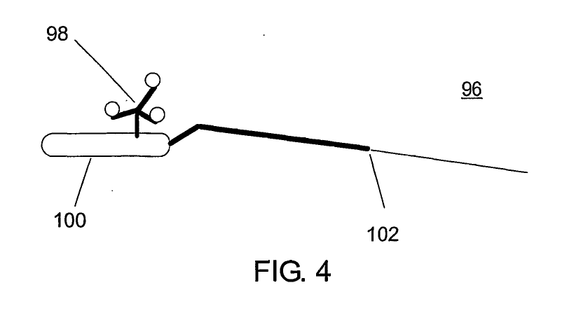

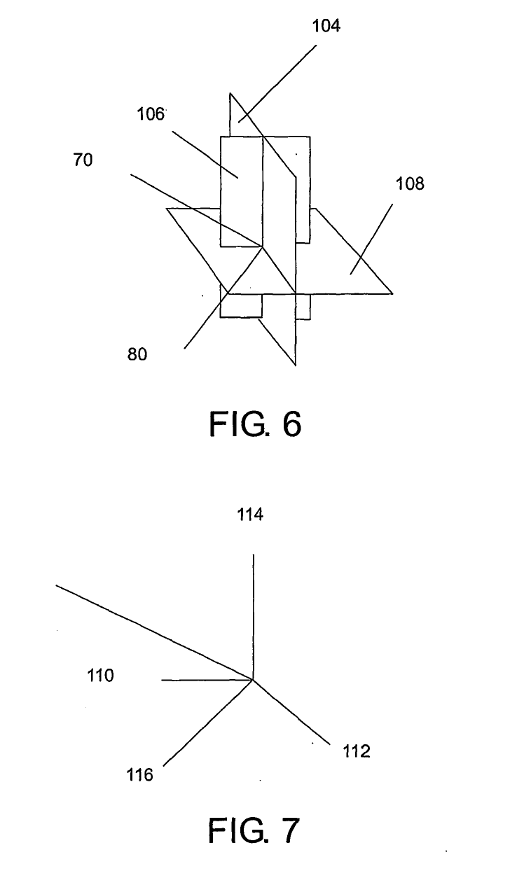

Referring to FIGS. 3 and 6, the frontal plane 104 is described by the x,y coordinates and the sagittal plane 106 by the x,z coordinates. Alpha 82 represents the inclination angle and gamma 84 the anteversion angle. The U 86 represents the tracker position of the tracked cup positioner 80, as identified by its spatial position (x,y,z). R 88 is the distance from the tracker on the tracked cup positioner 80 to the instrument tip. The distance R 88 is determined prior to intra-operative use on calibration of the tracked cup positioner 80. The origin of the coordinate system is defined to be the centre of the pubic symphysis landmark 70.

Both the origin of the coordinate system and U(x,y,z) are registered by the navigation software, allowing the calculation of both angles alpha 82 and gamma 84 using the following relationships:

Ux=R*sin(gamma)*cos(alpha)

Uy=R*sin(gamma)*sin(alpha)

Uz=R*cos(gamma)

R=sqr (Ux²+Uy²+Uz²)

The angle alpha 82 is the cup abduction angle, which is also the azimuth of U (spherical coordinate). The relationship between alpha 82 and the position of the tracked cup 80 is described as follows:

Alpha=arctan (Uy, Ux)

with arctan(y,x) defined as:

if x>0: tan⁻¹(y/x)

if x<0: pi+tan⁻¹(y/x)

if (x=0) and (y>0): pi/2

if (x=0) and (y<0): −pi/2

The angle gamma 84 is the cup anteversion angle, which is also the colatitude of U (spherical coordinate). The relationship between gamma 84 and the position of the tracked cup 80 is described as follows:

gamma=cos⁻¹ (Uz/R)

Referring to FIG. 6, the frontal 104, sagittal 106 and axial 108 planes form the pelvis coordinate system. FIG. 7 depicts the same coordinate system using the plane normals to represent the coordinate system for mathematical purposes. The normal of the frontal plane (n_{f}) 110, the normal of the sagittal plane (nₛ) 112 and the normal of the axial plane (nₐ) 114 are identified.

To determine the abduction angle 82 and the anteversion angle 84 for the left hip 78, first the direction vector v₀ of the cup positioner 80 is determined:

v₀=R*[001]

Next, the projection of the cup positioner out each plant normal is found:

Projection onto n_{f}=v_{cf}=(v_{O}*n_{f})n_{f}

Projection onto nₛ=v_{cs}=(v_{O}*nₐ)nₛ

Projection onto nₐ=v_{ca}=(v_{O}*nₐ)nₐ

Next the projection of the cup positioner onto each plane is found:

Projection onto frontal plane v_{f}=v_{cs}+v_{ca}

Projection onto sagittal plane vₛ=v_{cf}+v_{ca}

Projection onto axial plane vₐ=v_{cs}+v_{cf}

Next, the angle between the cup positioner vector (v_{O}) and each plume is found:

Angle cup positioner to frontal a_{f}=(180/π)cos(vₒ*v_{f})⁻¹

Angle cup positioner to sagittal plane aₛ=(180/π)cos(vₒ*vₛ)⁻¹

Angle cup positioner to axial plane aₐ=(180/π)cos(vₒ*vₐ)⁻¹

where the anteversion angle 84 is a_{f} and the abduction angle 82 is aₐ.

For the right hip 76, the negative of the abduction angle aₐ is used.

The computation of the angles alpha 82 and gamma 84 can be conducted within navigation software or the like and information regarding the trajectory of the tracked cup positioner 80, as well as the angles of approach with reference to alpha 82 and gamma 84, displayed on the computer display 44.

As will be appreciated by persons skilled in the art, the above may be adapted to ascertain the trajectory, including path, position and angle of approach, for any object relative to any anatomical structure, including skeletal structures, joints, soft tissue, organs, etc., for which landmarks to define a three-dimensional coordinate system for the anatomical structure can be identified.

As will also be appreciated by persons skilled in the art, while the above has been largely described with reference to a THR surgical procedure, the use of landmarks on anatomical structures to define a three-dimensional coordinate system has application in other computer-assisted surgical systems including diagnostic techniques and surgical planning.

Numerous modifications, variations, and adaptations may be made to the particular embodiments of the invention described above without departing from the scope of the invention, which are defined in the claims.

What is claimed is:

1. A method of defining a coordinate system for computer-assisted surgical systems, the method comprising the steps of:
identifying at least three landmarks associated with an anatomical structure;
determining a location for each of the landmarks;
refining the location for one or more landmarks using imaging; and
defining the coordinate system using the refined location for each of the landmarks.

2. A method of claim 1 wherein the step of determining a location for each of the landmarks includes determining a coordinate position for each of the landmarks for use in the computer-assisted surgical system.

3. A method of claim 2 wherein the step of determining the location of each of the landmarks includes using a location digitizing device.

4. A method of claim 1 wherein the step of refining the location for each of the landmarks includes using a single X-ray image in the plane of the image.

5. A method of claim 4 wherein the X-ray view is an anterior-posterior view.

6. A method of claim 4 wherein the X-ray view is a lateral view.

7. A method of claim 4 wherein more than one X-ray images are used in bi-planar X-ray reconstruction.

8. A method of claim 1 wherein the step of refining the location for each of the landmarks includes using ultrasound imaging.

9. A method of claim 1 wherein the step of defining the coordinate system comprises the steps of:
defining a first plane using the landmarks;
defining a second plane orthogonal to the first plane; and
using the planes for defining the coordinate system.

10. A method of claim 9 wherein the first plane is a frontal plane.

11. A method of claim 9 wherein the second plane is a sagittal plane.

12. A method of claim 9 further including the step of defining a third plane orthogonal to each of the first plane and the second plane.

13. A method of claim 12 wherein the third plane is an axial plane.

14. A method of claim 1 wherein the anatomical structure is a pelvis.

15. A method of claim 14 wherein the landmarks include a left anterior superior iliac spine, a right anterior superior iliac spine, and a centre of a public symphysis.

16. A method of claim 15 wherein the frontal plane is defined using the left anterior superior iliac spine, the right anterior superior iliac spine, and the centre of the pubic symphysis.

17. A method of claim 16 wherein the centre of the pubic symphysis is centred in the anterior posterior plane.

18. A method of claim 1 wherein the step of defining the coordinate system using the refined location for each of the landmarks includes the step of defining at least two vectors using the refined location for each of the landmarks.

19. A method of claim 18 wherein the step of defining at least two vectors includes the steps of:
defining a first vector using at least two landmarks; and
defining a second vector using at least two landmarks, said second vector being non-parallel to the first vector.

20. A method of claim 19 wherein the first vector is defined using the left anterior superior iliac spine and the right anterior superior iliac spine.

21. A method of claim 20 wherein the second vector is defined using the first vector and the centre of the pubic symphysis.

22. A method of claim 19 wherein the first vector and the second vector are used in defining the frontal plane and the sagittal plane.

23. A method of any one of claims 1 to 22 wherein the coordinate system is used for ascertaining the trajectory of an object with reference to the anatomical structure.

24. A method of claim 23 wherein the object is a tracked instrument.

25. A method of claim 24 wherein the object is an acetabular cup positioner.

26. The use of a coordinate system defined in any one of claims 1 to 25, intra-operatively.

27. The use of a coordinate system of claim 26 in surgery.

28. The use of a coordinate systems of claim 27 in total hip replacement surgery.

29. A system for defining a coordinate system for computer-assisted surgical systems, the system comprising:
means for determining a location for each of at least three landmarks associated with an anatomical structure;
means for refining the location for each of the landmarks using imaging; and
means for defining the coordinate system using the refined location for each of the landmarks.

30. A system of claim 29 further including means for defining a first plane using the landmarks; means for defining a second plane orthogonal to the first plane; and means for using the planes for defining the coordinate system.

31. A system of claim 30 further including means for ascertaining the trajectory of an object in relation to an anatomical structure for use with computer-assisted surgery.

32. A system of claim 29 wherein means for defining the coordinate system using the refined location for each of the landmarks includes means for defining at least two vectors using the refined location for each of the landmarks.

33. A system of claim 32, wherein means for defining at least two vectors includes the steps of:
defining a first vector using at least two landmarks; and
defining a second vector using at least two landmarks, said second vector being non-parallel to the first vector.

34. A computer-readable medium having computer-executable software code stored thereon, comprising:
a code for determining a coordinate position for each of at least three landmarks identified, wherein the landmarks are associated with the anatomical structure;
a code for refining the coordinate position for each of the landmarks; and
a code for defining the coordinate system using the refined coordinate positions for each of the landmarks.

35. A computer-readable medium of claim 32 further including:
a code for defining a first plane using the landmarks;
a code for defining a second plane orthogonal to the first plane; and
a code using the planes for defining the coordinate system.

36. A computer-readable medium of claim 33 further including:
a code for ascertaining the trajectory of an object with reference to the anatomical structure for using the coordinate system.

37. A system according to any one of claims 29 to 33 wherein the means for determining a location for each of the landmarks includes a C-arm device.
